(19) United States
(12) Patent Application Publication
(10) Pub. No.: US 2015/0086587 A1
(43) Pub. Date: Mar. 26, 2015

(54) VACCINE ADJUVANT

(71) Applicants: Kansas State University Research Foundation, Manhattan, KS (US); University Of Tennessee Research Foundation, Knoxville, TN (US)

(72) Inventors: Jishu Shi, Manhattan, KS (US); Loretta Pappan, Rougemont, NC (US); Amy Beckley, Manhattan, KS (US); Qixin Zhong, Knoxville, TN (US)

(21) Appl. No.: 14/388,422

(22) PCT Filed: Mar. 28, 2013
(86) PCT No.: PCT/US2013/034372

§ 371 Date: Sep. 26, 2014

Related U.S. Application Data
(60) Provisional application No. 61/616,681, filed on Mar. 28, 2012.

(51) Int. Cl. A61K 39/39; A61K 39/145; A61K 39/02

(52) U.S. Cl. CPC A61K 39/39; A61K 39/0241; A61K 39/145; A61K 2039/55588
USPC 424/209.1; 424/283.1; 424/264.1

(57) ABSTRACT

Oil-based adjuvant emulsions, immunogenic compositions, and methods of using the same to elicit immunological responses in a subject are provided. The oil-based adjuvants comprise a plant-derived surfactant, such as gum arabic, an aqueous component, and an oil. The adjuvant emulsions can be used to potentiate the immunological effects of active agents, including antigens derived from various pathogens and/or toxins. Immunogenic compositions comprising the adjuvant emulsions and various active agents are also provided, along with vaccine systems for therapeutic or prophylactic treatment of a subject using these adjuvants.

### CROSS-REFERENCE TO RELATED APPLICATIONS

The present application claims the priority benefit of U.S. Provisional Patent Application Ser. No. 61/616,681, filed Mar. 28, 2012, entitled Vaccine Adjuvant, incorporated by reference in its entirety herein.

### BACKGROUND OF THE INVENTION

1. Field of the Invention

The present invention relates to plant-derived emulsifiers as adjuvants, associated vaccines, and pharmaceutical or veterinary compositions containing the same.

2. Description of Related Art

Adjuvant has been used for decades to improve the immune response to vaccine antigens. An adjuvant is a substance that increases the immunological response to a vaccine when administered before, during, or after administration of the vaccine. Adjuvants potentiate the vaccine by stimulating antigen-presenting cells and other immune cells or by controlling the release of antigens from the injection site. Adjuvant can not only reduce the antigen dose used, but also increase immune duration. An adjuvant may be administered with the vaccine or at a time, manner, or site that differs from the time, manner, or site at which the vaccine is administered. Vaccines containing dead organisms (inactivated vaccines) or pieces of the infectious organisms or their toxins (acellular or recombinant vaccines) generally need adjuvants to boost their effectiveness. Common adjuvants include aluminum hydroxide, aluminum potassium sulfate, other mineral salts, oil emulsions (e.g., squalene), particulate adjuvants, and microbial derivatives. Many existing adjuvants have unacceptable side effects and lack biocompatibility. For example, aluminum-based adjuvants, such as alum and aluminum hydroxide, are the most commonly used adjuvant in the United States. However, they are also known to cause injection site reaction. Conventional oil-in-water emulsions use various chemical emulsifiers or surfactants, such as TWEEN® 80 (polyoxyethylene sorbitan monooleate) and Span 80 (sorbitan trioleate), which may also have biocompatibility issues.

New vaccine candidates have been developed over the past years against infectious, allergic, and autoimmune diseases and also for cancer and fertility treatments. All of these applications require adjuvants with desirable functions and performance in order to successfully achieve new vaccine development and implementation. Thus, there remains a need in the art for adjuvants having improved performance, biocompatibility, and lower cost.

### SUMMARY OF THE INVENTION

The present invention is broadly concerned with novel adjuvants, immunogenic compositions, and methods of using the same to elicit immunological responses in a subject. In one aspect, an oil-based adjuvant comprising a plant-derived surfactant, an aqueous component, and an oil is provided. Nontoxic plant-derived surfactants, such as gum arabic are particularly preferred for use in the invention.

An immunogenic composition for eliciting an immunological response is also described herein. The immunogenic composition comprises an adjuvant emulsion, as described herein, and an active agent, such as an antigen derived from a pathogen and/or its cells, tissues, toxins, subunits, particles, nucleic acids, and/or surface proteins. Other active agents include killed virus, modified live virus, viral or bacterial proteins, viral or bacterial DNA, toxoids, protein subunits, tumor antigens, and combinations thereof.

The present disclosure also describes methods of eliciting an immunological response in a subject. The method comprises administering to the subject an adjuvant and an active agent as described herein. The methods can be used for both therapeutic and/or prophylactic purposes.

Also described herein is a vaccine system comprising an adjuvant as described herein and instructions for co-administering the adjuvant to a subject with an active agent.

### BRIEF DESCRIPTION OF THE DRAWINGS

FIG. 1 is a graph of the ELISA optical density (OD) values for the oil-based adjuvanted vaccines formulated in Example 2;

FIG. 2 is a graph showing the haemagglutination inhibition (HI) titers from vaccinating swine with formulation 12b in Example 4;

FIG. 3 is a graph of the mycoplasma antibody titers from vaccinating swine with formulation 12b in Example 4;

FIG. 4 is a graph of the mycoplasma antibody titers from vaccinating swine with formulation 14b in Example 4 based on different amounts of antigen used in the vaccine;

FIG. 5 is a graph of the mycoplasma antibody titers over time from vaccinating swine with formulation 14b in Example 4;

FIG. 6 is a transmission electron micrograph (TEM) image (at 130,000×mag) of adjuvant emulsion 14 showing sizes of some of the stabilized droplets; and

FIG. 7 is a TEM image (at 34,000×mag) of adjuvant emulsion 14 showing sizes of some of the stabilized droplets.

### DETAILED DESCRIPTION OF PREFERRED EMBODIMENTS

The present invention is concerned with new oil-based adjuvant compositions comprising (consisting essentially or even consisting of) an oil and a plant-derived surfactant. The term “adjuvant” is used herein to refer to substances that have immunopotentiating effects and are added to or co-formulated with an active agent in order to enhance, induce, elicit, and/or modulate the immunological response against the active agent when administered to a subject. In one or more embodiments, the adjuvant compositions are in the form of emulsions comprising (consisting essentially or even consisting of) the plant-derived surfactant, along with an aqueous component and an oil component, and more specifically can be in the form of water-in-oil, oil-in-water, or water-in-oil-in-water emulsions. In one or more embodiments, the adjuvant compositions are in the form of dried (e.g., freeze or spray dried) compositions of the plant-derived, surfactant-stabilized oil. The dried adjuvant compositions can be reconstituted in an appropriate aqueous component to create an emulsion for use with a vaccine system. In one or more embodiments, the plant-derived surfactant creates a stabilizing layer or “shell” around oil droplets in the emulsion. The plant-derived surfactant not only stabilizes these oil-based emulsions, but improves the immunopotentiating effects of the adjuvant emulsion.

The plant-derived surfactant is a natural or synthetic compound obtained from a plant (it's tissues, cells, roots, etc.), whether through chemical or biological processes, which acts as a surface active agent by promoting the formation (dispersion) and stabilization of an emulsion. Nontoxic plant-derived surfactants are particularly preferred, and expressly exclude saponins and their derivatives. In one or more embodiments, the plant-derived surfactant is a plant-derived polysaccharide. In one or more embodiments, the plant-derived surfactant is a water-soluble, plant-derived gum. Examples of suitable plant-derived surfactants include gum arabic, soluble soybean polysaccharides, pectins (e.g., sugar beet-sources), cellulose derivatives (e.g., hydroxypropyl methylcellulose), modified starch, plant protein conjugates, conjugates prepared from reducing saccharides, derivatives thereof, and the like. In one or more embodiments, the plant-derived surfactant is gum arabic or a derivative thereof. The plant-derived surfactant can be used in the adjuvant composition at a level of from about 0.001% to about 20% by weight, preferably from about 0.1% to about 10% by weight, and more preferably, from about 0.1% to about 5% by weight, based upon the total weight of the adjuvant emulsion taken as 100% by weight.

The aqueous component is typically unadulterated water, such as ultrapure water, nanopure water, double-distilled water, sterile water/distilled autoclaved water (DAW), and the like, but may also include buffered aqueous solutions, such as buffered saline (e.g., phosphate buffered saline), cell culture medium, and the like. The aqueous component can be used in the adjuvant composition at a level of from about 30% to about 99.999% by weight, preferably from about 70% to about 99% by weight, and more preferably, from about 80% to about 95% by weight, based upon the total weight of the adjuvant emulsion taken as 100% by weight.

The oil component can be a metabolizable or non-metabolizable oil. Oils derived from plant or animal sources can be used in the invention, as well as petroleum derivatives and synthetic oils. Plant oils include vegetable oils from the seeds, nuts, or grains of a plant, such as soybean oil, sunflower oil, canola oil, olive oil, cottonseed oil, palm oil, corn oil, rice bran oil, sorghum oil, and the like. Animal-derived oils include squalene, milk fat, and the like. Petroleum derivatives and synthetic oils include mineral (paraffin) oil and the like. Mixtures of oils may also be used. The oil component can be used in the adjuvant composition at a level of from about 0.001% to about 70% by weight, preferably from about 1% to about 30% by weight, and more preferably, from about 5% to about 20% by weight, based upon the total weight of the adjuvant emulsion taken as 100% by weight.

The adjuvant compositions can also contain antibiotics and/or preservatives, including, for example, gentamicin, formaldehyde, amphotericin, and the like. When present, the adjuvant compositions will comprise from about 0.02% to about 2% by weight antibiotics, preferably from about 0.02% to about 0.2% by weight antibiotics, and more preferably, from about 0.02% to about 0.1% by weight antibiotics, based upon the total weight of the adjuvant emulsion taken as 100% by weight. When present, the adjuvant compositions will comprise from about 0.01% to about 1% by weight preservatives, preferably from about 0.01% to about 0.5% by weight preservatives, and more preferably, from about 0.01% to about 0.1% by weight preservatives, based upon the total weight of the adjuvant emulsion taken as 100% by weight.

If desired, the adjuvant compositions can further comprise one or more additional ingredients, such as co-adjuvants, other stabilizers and/or emulsifiers, and the like. Exemplary co-adjuvants for use in oil-based emulsions include block copolymers, bacteria-derived lipids and/or cell wall components, mineral salts, cytokines, cytokine-inducing agents, and the like. In one or more embodiments, the adjuvant compositions are substantially free of co-adjuvants. Exemplary stabilizers and/or emulsifiers that can be included in the adjuvant compositions include oleates, saponins, co-polymers, and the like. In one or more embodiments, the adjuvant compositions are substantially free of any other stabilizers and/or emulsifiers (other than the plant-derived surfactants described above). For example, in some embodiments, the adjuvant compositions are substantially free of oleates, such as polysorbate 80, sorbitan trioleate, glycerol monooleate, co-polymers, and the like. In some embodiments, the adjuvant compositions are substantially free of saponins. The term “substantially free,” as used herein, means that the ingredient is not intentionally added to the composition, although incidental impurities may occur. In such embodiments, the adjuvant compositions comprise less than about 0.05% by weight, preferably less than about 0.01%, and more preferably about 0% by weight of such an ingredient, based upon the total weight of the emulsion taken as 100% by weight.

The adjuvant composition can be prepared by first dissolving or dispersing the plant-derived surfactant in the aqueous component (or a portion thereof). If desired, the resulting surfactant solution can be allowed to stand for a time period of up to about 24 hours. The surfactant solution is then mixed with the oil component until a substantially homogenous dispersion is formed. It will be appreciated that vigorous mixing may be required to adequately disperse the oil and aqueous components to achieve the adjuvant emulsion. In some embodiments, the components can be mixed together under high shear using a high shear mixer, such as a high shear fluid processor. Additional processes that can be used to improve stability or change emulsion characteristics, such as ultrasonication, high pressure homogenization, colloid mills, and microfluidization. As noted above, additional ingredients can be added to the adjuvant composition. In some embodiments, the additional ingredients will be added before emulsifying the composition. In other embodiments, the ingredients can be added after emulsifying the composition. The resulting adjuvant emulsion can then be stored at temperatures ranging from about −80° C. to about 50° C. until use. Advantageously, the emulsions are shelf-stable at room temperature (˜25° C.) for a time period of at least about 12 months, and shelf-stable at 4° C. for at time period of at least about 24 months. The term “shelf-stable” means that when stored at the indicated temperature, the emulsions do not separate into their constituent parts and the oil component does not come out of solution.

It will be appreciated that the resulting adjuvant emulsions can also be sterilized by heat or alternative techniques, such as ultraviolet radiation for storage without refrigeration or freezing. Alternatively, the adjuvant emulsions can be prepared in powdered (dried) form by processes such as spray drying of freeze drying.

Adjuvants according to the invention are useful for potentiating the immune effects of immunogenic compositions (e.g., vaccines) and other pharmaceutical or veterinary compositions. Thus, the adjuvants may be co-administered with such active agents. The term “co-administer,” as used herein, is intended to embrace separate administration of the adjuvant and active agent in a sequential manner as well as co-administration of these agents in a substantially simultaneous manner, such as in a single mixture/composition or in doses given separately, but nonetheless administered substantially simultaneously to the subject.

In one or more embodiments, the invention is also concerned with an immunogenic composition for eliciting an immunological response comprising (consisting essentially or even consisting of) an adjuvant as described above and an active agent. In one or more embodiments, the immunogenic composition comprises the adjuvant emulsion with the plant-derived surfactant-stabilized oil droplets and the active agent dispersed in the aqueous component (i.e., outside of the oil droplets and not encapsulated by them). The active agent can be any of a wide variety of substances capable of generating (directly or indirectly) a desired immune response in a subject. In general, the active agent useful in the inventive embodiments is an immunogenic active component (e.g., antigen) in that it resembles a disease-causing pathogen (e.g., microorganism) or infectious agent, and/or is made from weakened, killed, and/or recombinant forms of the same, its cells, tissues, toxins, subunits, particles, and/or one of its surface proteins, such that it provokes an immune response to that pathogen or infectious agent.

The active agent may comprise a killed, but previously virulent, microorganism that has been destroyed. Examples of such active agents are found in influenza, cholera, polio, hepatitis A, and rabies vaccines. Other active agents may contain live, attenuated microorganisms (modified live virus), which either use live viruses that have been cultivated under conditions that disable their virulent properties, or use closely related but less dangerous organisms to produce a broad immune response. Examples include yellow fever, measles, mumps, rubella, whooping cough, porcine reproductive and respiratory syndrome (PRRS), distemper, canine adenovirus Type 2, parainfluenza, and kennel cough (e.g., coronavirus) vaccines. Some active agents are also bacterial in nature, such as Mycoplasma hyopneumoniae, Bordetella bronchiseptica, Mycobacterium tuberculosis, Salmonella typhi (typhoid), Staphylococcus aureus, and Streptococcus suis. Other suitable active agents include toxoids made from inactivated toxic compounds that cause illness rather than the microorganism itself Examples of toxoid-based vaccines include tetanus and diphtheria. Protein subunit active agents can also be used, in which a fragment of the microorganism is used to create an immune response. Examples include subunit vaccines against HPV, hepatitis B, and the hemagglutinin and neuraminidase subunits of the influenza virus. Active agents can also be formulated using viral or bacterial DNA to provoke an immune response. Furthermore, although most current vaccines are created using inactivated or attenuated compounds from microorganisms, synthetic active agents using synthetic peptides, carbohydrates, or antigens can also be used. Cancer vaccines using tumor antigens as the active agent are also contemplated herein. Suitable immunogenic compositions can be monovalent or polyvalent.

It will be appreciated that the amount of active agent present in the immunogenic compositions will vary depending upon the particular active agent used, as well as the intended subject for administration of the composition, and route of administration. In general, the immunogenic compositions will comprise from about 0.05% to about 10% by weight active agent, preferably from about 0.05% to about 1% by weight active agent, and more preferably, from about 0.05% to about 0.5% by weight active agent, based upon the total weight of the immunogenic composition taken as 100% by weight. The immunogenic compositions will also comprise from about 0.5% to about 70% by weight adjuvant, preferably from about 0.5% to about 7% by weight adjuvant, and more preferably, from about 0.5% to about 3.5% by weight adjuvant, based upon the total weight of the immunogenic composition taken as 100% by weight. It will be appreciated that suitable carriers or diluents for the active agent, along with any of the optional ingredients noted above will account for the remaining portion of the immunogenic compositions. In some embodiments, the weight ratio of adjuvant to active agent in the composition will be from about 10:1 to about 40:1, preferably from about 20:1 to about 40:1, and more preferably from about 30:1 to about 40:1.

Additional ingredients that can be included in the immunogenic compositions include carriers, preservatives, solubilizing agents, anesthetics, and the like. For example, a local anesthetic (e.g., lidocaine) may be included in the compositions to ease pain at the site of the injection for injectable forms of the immunogenic compositions. Likewise, active agents are often provided as desiccated or freeze-dried preparations of the antigenic substrate that can be rehydrated by dispersing in a carrier or diluent before being mixed with the adjuvant. Alternatively, the active agent may be rehydrated directly in the inventive adjuvant emulsion. In some embodiments, the active agents can be provided pre-dispersed in a carrier, and this active agent dispersion can be directly mixed with the adjuvant. In other embodiments, it may be desirable to separate or filter the active agent from the carrier before mixing the active agent into the adjuvant emulsion. The term “carrier” is used herein to refer to diluents, excipients, vehicles, and the like, in which the active agent(s) may be dispersed for administration. Suitable carriers will be pharmaceutically acceptable. As used herein, the term “pharmaceutically acceptable” means not biologically or otherwise undesirable, in that it can be administered to a subject without excessive toxicity, irritation, or allergic response, and does not cause any undesirable biological effects or interact in a deleterious manner with any of the other components of the composition in which it is contained. A pharmaceutically-acceptable carrier or excipient would naturally be selected to minimize any degradation of the active agent or adjuvant and to minimize any adverse side effects in the subject, as would be well known to one of skill in the art. Pharmaceutically-acceptable ingredients include those acceptable for veterinary use as well as human pharmaceutical use. Carriers suitable for administration via injection are typically solutions in sterile isotonic aqueous buffer. Exemplary carriers and excipients include aqueous solutions such as normal (n.) saline (˜0.9% NaCl), phosphate buffered saline (PBS), DAW, as well as cell growth medium (e.g., MEM, with or without serum,), aqueous solutions of dimethyl sulfoxide (DMSO), polyethylene glycol (PEG), and/or dextran (less than 6% per by weight.), various oil-in-water or water-in-oil emulsions, and the like.

The adjuvants, active agents, and associated immunogenic compositions are useful in eliciting an immunological response in a subject. In use, a therapeutically-effective amount of adjuvant, active agent, or associated immunogenic compositions is administered to a subject. As used herein, a “therapeutically-effective” amount refers to the amount that will elicit the biological or medical response of a tissue, system, or subject that is being sought by a researcher or clinician, and in particular elicit some desired therapeutic or prophylactic effect. One of skill in the art recognizes that an amount may be considered therapeutically effective even if the condition is not totally eradicated or prevented but improved or inhibited partially. Advantageously, use of the novel plant-derived adjuvant emulsion may decrease the therapeutically-effective amount of active agent required to elicit the desired immunological response in the subject.

Immunogenic compositions according to the invention can have prophylatic and/or therapeutic uses. The terms “therapeutic” or “treat,” as used herein, refer to processes that are intended to produce a beneficial change in an existing condition (e.g., infection, disease, disorder) of a subject, such as by reducing the severity of the clinical symptoms and/or effects of the infection, and/or reducing the duration of the infection/symptoms/effects. Thus, in some embodiments, the subject is afflicted with a condition, wherein methods described herein are useful for treating the condition and/or ameliorating the effects of the condition. In other embodiments, the subject is free of a given condition, wherein the methods described herein are useful for preventing the occurrence of the condition and/or preventing the effects of the condition. The terms “prophylactic” or “prevent,” as used herein, refer to processes that are intended to inhibit or ameliorate the effects of a future condition that a subject may incur (but is not necessarily suffering with presently). In some cases the composition may prevent the development of observable morbidity from the condition (i.e., near 100% prevention). In other cases, the composition may only partially prevent and/or lessen the extent of morbidity due to the condition (i.e., reduce the severity of the symptoms and/or effects of the infection, and/or reduce the duration of the infection/symptoms/effects). In either case, the compounds are still considered to “prevent” the target condition.

The disclosed embodiments are suitable for various routes of administration. The adjuvants, active agents, and/or associated immunogenic compositions can be injected intramuscularly, subcutaneously, intradermally, or intraperitoneally. They can also be administered orally or intranasally.

A vaccine system or kit comprising the novel adjuvant and instructions for using the adjuvant with an active agent is also disclosed herein. The kit may further comprise active agents, instructions for preparing an immunogenic composition, as well as instructions for administering the adjuvant and active agent to a subject.

The adjuvant and active agent components of the immunogenic compositions can be supplied either separately or pre-mixed together in a unit dosage form. The term “unit dosage form” refers to a physically discrete unit suitable as a unitary dosage for human or animal use. Each unit dosage form may contain a predetermined amount of the active agent (and/or adjuvant) calculated to produce a desired effect. In one or more embodiments, the plant-derived adjuvant emulsion can be provided separately from the active agent (e.g., in its own vial, ampule, sachet, or other suitable container). Likewise, the active agent can be provided separately from the plant-derived adjuvant emulsion (e.g., in its own container). In some embodiments, additional ingredients, such as suitable carriers, can be present in the active agent container. In other embodiments, any additional ingredients can be provided in yet a separate container. Regardless, when the ingredients are separately provided, it will be appreciated that the ingredients can then be mixed onsite by a practitioner to create the immunogenic composition before being administered to the subject.

It will be appreciated that therapeutic and prophylactic methods described herein are applicable to humans as well as any suitable animal, including, without limitation, dogs, cats, and other pets, as well as, rodents, non-human primates, horses, cattle, pigs, birds, etc. The methods can be also applied for clinical research and/or study. Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Plant-Derived Surfactants as Oil and Water Stabilizers/Emulsifiers

To determine of if plant-derived surfactants improved the stability of oil and water emulsions, emulsifier solutions of soybean soluble polysaccharide (SSPS), gum arabic, and hydroxypropyl methylcellulose (HPMC) were prepared. A 0.2% SSPS solution was prepared by adding 0.2 g of SSPS to 99.8 ml double distilled water (ddH₂O), followed by stirring gently until the SSPS went into solution, with additional stirring for about 60 minutes. A 1% gum arabic solution was prepared by adding 1 g of gum arabic to 99 ml of ddH₂O, and stirring rapidly into solution, followed by additional stirring for about 60 minutes. A 2% HPMC solution was prepared by adding 2 g of HPMC2906 (METHOCEL® F; Dow Chemical) to 20 ml of heated ddH₂0 until completely wetted, followed by adding 78 ml ddH₂0 to the wetted HPMC, and stirring for about 90 minutes.

Next, 10-90 mL of the above plant-derived surfactant solutions were added to 10-20 mL of mineral oil to produce 10-20% oil emulsions. The emulsions were then stored at room temperature or 4° C. for a period of over 3 months and periodically observed. The emulsions remained stable, without any oil falling out of solution for over 3 months and without the mixtures separating into their constituent parts. The results indicate that plant-derived surfactants can be used as emulsifiers in oil/water solutions.

### Example 2

### Vaccination Using Plant-Derived Surfactants as Adjuvants

### Adjuvant and Vaccine Preparation

In this Example, various adjuvant emulsions and vaccine compositions were prepared. Adjuvant emulsions were first prepared using either gum arabic or HPMC as the plant-derived surfactant in the emulsion. The adjuvant emulsions were the mixed with various amounts of antigen to create 2 mL dosages for vaccination. The vaccines in category “a” were mixed in a 1:1 ratio of adjuvant emulsion (1 mL) to mycoplasma antigen (1 mL, containing inactivated bacterin Mycoplasma hypopneumoniae with amphotericin B and gentamicin as preservatives; Newport Laboratories, Worthington, Minn.). The vaccines in category “b” were mixed in a 1:1:1 ratio of adjuvant emulsion (0.667 mL) to mycoplasma antigen (0.667 mL) to H3N2 antigen (0.667 mL, containing inactivated Swing Influenza virus (viral titer 32 HAU/50 μL) and amphotericin B and gentamicin as preservatives; Newport Laboratories).

TABLE
Vaccine Formulations

| Vaccine | Surfactant | Oil | Antigen |
|---|---|---|---|
| 1a | 0.5% HPMC | 10% | mycoplasma |
| 1b | 0.333% HPMC | 6.67% | mycoplasma & H3N2 |
| 2a | 0.5% HPMC* | 10% | mycoplasma |
| 2b | 0.333% HPMC* | 6.67% | mycoplasma & H3N2 |
| 3a | 1.25% gum arabic | 10% | mycoplasma |
| 3b | 0.833% gum arabic | 6.67% | mycoplasma & H3N2 |

*Different brand of HPMC than used in vaccine 1.

### Vaccination

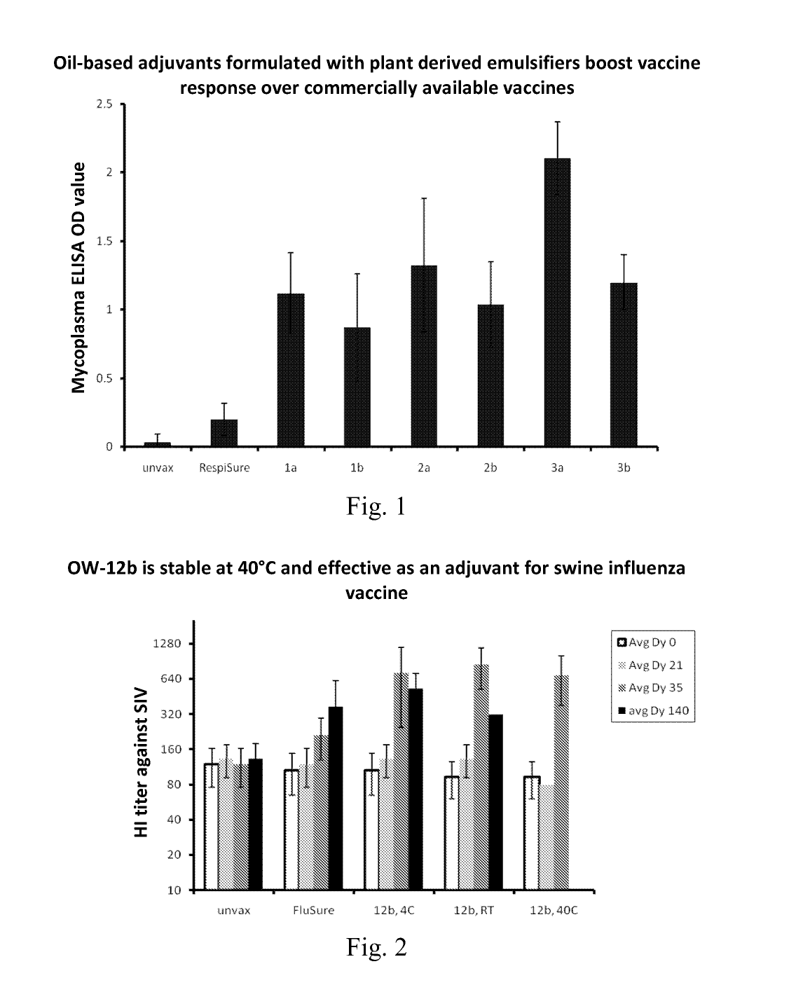

The prepared vaccines were then injected intramuscularly into pigs at 3 weeks of age (Day 0). An unvaccinated group was used as a control. Another group of pigs was vaccinated using a commercially-available mycoplasma vaccine (Respisure®; chemically inactivated whole cell culture of M. hyopneumoniae, and oil adjuvant, Amphigen®; Zoetis). Sera were drawn at Day 35 post-vaccination to measure the antibody response. Anti-mycoplasma antibody titer was determined using ELISA by the Diagnostic Laboratory of the College of Veterinary Medicine at Iowa State University. The OD values of serum samples in the ELISA were used for the comparison of immune responses from different treatment groups. The results are shown in FIG. 1. The use of the plant-derived, oil-based adjuvants improved the immunological response over the commercially-available vaccine.

### Example 3

### Adjuvant and Vaccine Preparation

### Adjuvant #12 (5% Gum Arabic, 10% Oil)

A 5% solution of Gum Arabic in nanopure water was prepared by adding 5 g of gum arabic to 85 mL of rapidly stirring water until dissolved. The solution was then allowed to stand at room temperature overnight to hydrate the polymer. Next, 10 mL of mineral oil was added to the hydrated polymer and mixed using a Silverson Lab mixer (L5M-A, Silverson, East Longmeadow, Mass.) for 15 minutes at 10,000 rpm. The adjuvant composition was passed five times through a Microfluidizer (M-110P, Microfluidics, Newton, Mass.) at ˜10,000 psi, and then the filtered composition was stored 4° C. To stop fungal and bacterial growth, 700 ppm formaldehyde and 30 μg/mL gentamicin were added to the composition.

### Vaccine #12b (2.5% Gum Arabic, 5% Oil)

Adjuvant #12, prepared above, was mixed 2:1:1 with antigen by adding 10 mL of the adjuvant emulsion, 5 mL of killed Swine Influenza Virus (H3N2; Newport Laboratories), and 5 mL of killed Mycoplasma hyopneumoniae (Newport Laboratories) to a tube. The tube was inverted several times to mix the solution and then stored at 4° C.

### Adjuvant #14 (7.5% Gum Arabic, 15% Oil)

A 7.5% solution of Gum Arabic in nanopure water was prepared using the same procedure described above, except that 7.5 g of gum arabic was added to 77.5 mL of water, followed by the addition of 15 mL of mineral oil.

### Vaccine #14b (2.5% Gum Arabic, 5% Oil)

A higher concentration vaccine was formulated, as described above, except that the adjuvant was mixed 1:1:1 with antigen using 10 mL of Adjuvant #14, 10 mL killed Swine Influenza Virus, and 10 mL of killed Mycoplasma hyopneumoniae.

### Stability

Adjuvants #12 and 14 were subjected to extended stability testing and stored for over 6 months. The adjuvant formulations stored at 40° C. were stable for over 2 weeks, and those stored at 4° C. and room temperature remained stable for more than 6 months.

For the vaccination studies, the adjuvants were stored at 4° C., room temperature (RT), or 40° C. for 2 weeks prior to be mixed with killed antigens. The formulated vaccines were then stored at 4° C., room temperature, or 40° C. for 10 days before vaccinating pigs, as described in Example 4.

### Example 4

### Adjuvanticity of Gum Arabic

In this example, the adjuvanticity of gum arabic was determined by immunizing swine with inactivated H3N2 Swine Influenza Virus and killed Mycoplasma hyopneumoniae in the presence or absence of gum arabic adjuvant.

### Animals and Vaccination Study

Pigs (female or male, 3 week old, 5-6 pigs per group) were immunized twice in a 3-week interval (at 3 and 6 weeks of age) with vaccines #12b and #14b formulated in Example 3 above. Negative controls received PBS, while the positive control group received a commercial vaccine (FluSure; Pfizer). Sera were collected from each pig to monitor antibody responses.

### Antibody Response Analysis

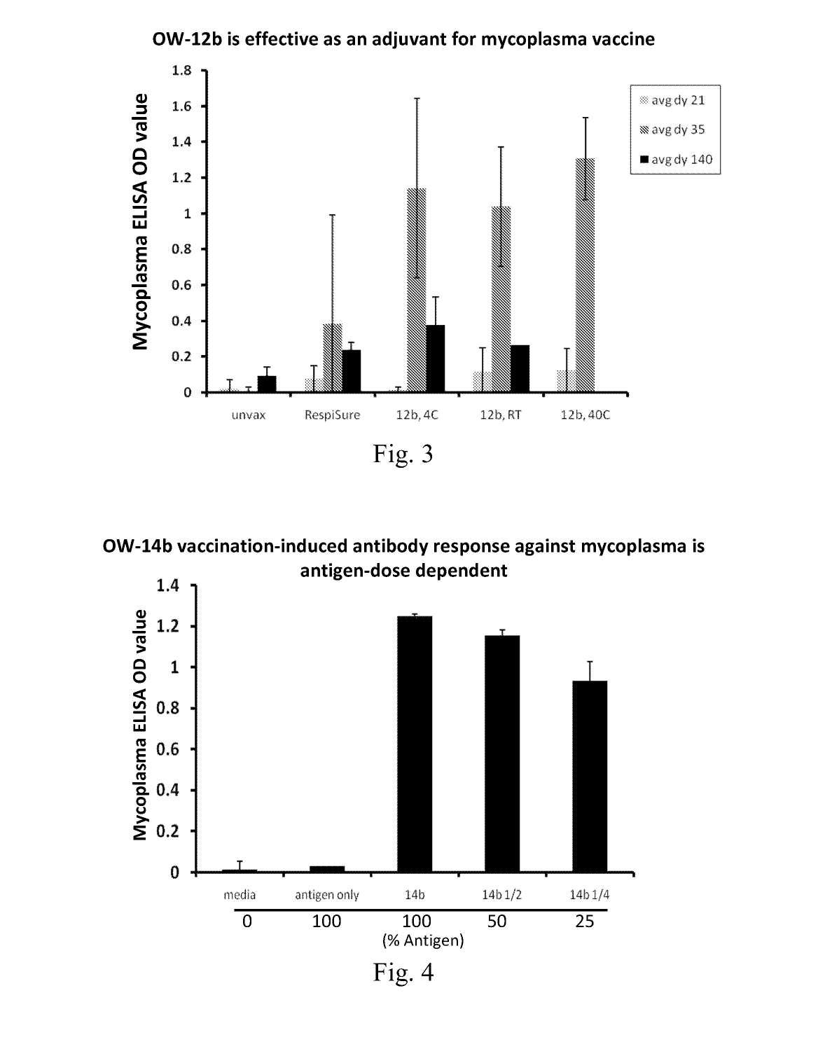

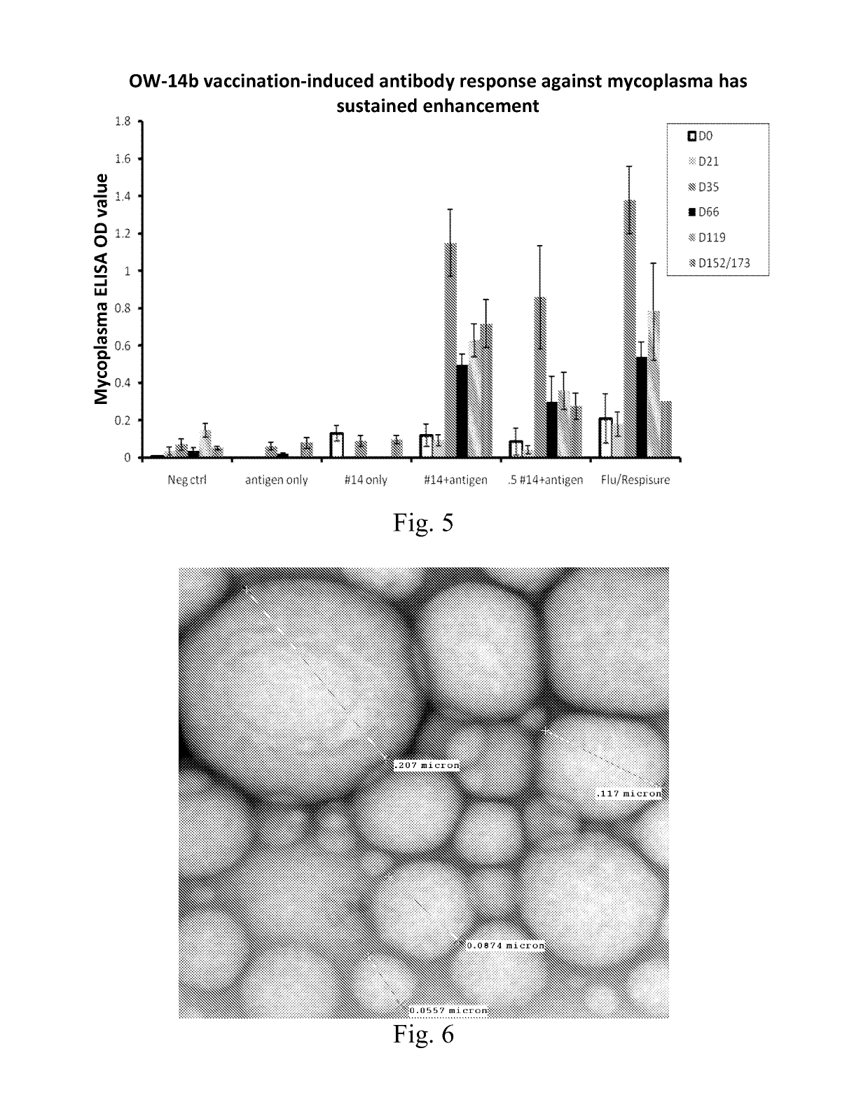

The haemagglutination inhibition (HAI) titers followed the WHO protocol. After proper treatments, heat-inactivated sera were serially diluted and preincubated at room temperature with 4 HA units/50 ml of H3N2 virus for 30 min. An equal volume of 0.5% chicken red blood cells was then added to each well and incubated at room temperature for 30 min. The HAI titer was read as the reciprocal of the highest dilution of serum that conferred inhibition of haemagglutination. The HAI titer results for vaccine #12b are shown in FIG. 2. The values were expressed as the geometric mean of each treatment group. Anti-mycoplasma antibody titer was determined using ELISA as described in Example 2 above. The results are shown in FIG. 3. In one study, differing amounts of antigen (Mycoplasma hyopneumoniae) were mixed with the adjuvant and the antibody response was tested by ELISA 42 days post vaccination. The results are shown in FIG. 4. The antibody response was followed until market age (Day 152). Pathology at necropsy (Day 173) found no injection site damage in any animals. The results are shown in FIG. 5.

### Discussion:

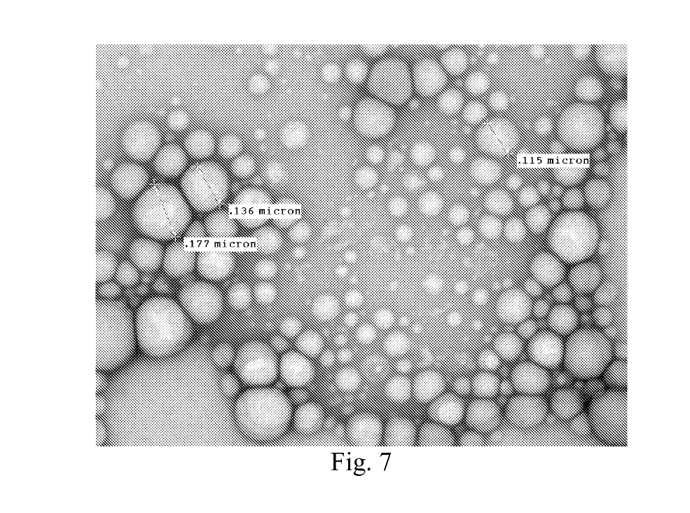

The adjuvant emulsions formulated in the present examples were very stable, as indicated by the stability tests above. The plant-derived surfactant seems to form a stabilizing “shell” around the oil droplets, maintaining them in size range of about 50 to about 200 nm in diameter. TEM images were taken of adjuvant emulsion #14 using an FEI Transmission Electron Microscope equipped with an AMT digital image capturing system, and are shown in FIGS. 6-7.

When mixed with the antigen, the hydrophilic antigen remains in the solution outside of the oil droplets. Various concentrations of emulsion #12 were formulated and analyzed using a particle analyzer. The mean hydrodynamic diameter of the oil droplets remained between about 220 and 240 nm, with a polydispersity of about 0.09 to about 0.15. Various concentrations of emulsion #14 were formulated and analyzed using a particle analyzer. The mean hydrodynamic diameter of the oil droplets remained between about 240 and 295 nm, with a polydispersity of about 0.106 to about 0.254. For both emulsions, the droplets were uniform and mono-dispersed. The diameter did not change significantly with different concentrations, indicating the droplets are stable in the measurement time scale.

The ability to induce SIV-specific and mycoplasma-specific antibody response of the gum Arabic adjuvant is similar as that of the commercial adjuvant. No injection site reaction (redness and swollen) was observed in vaccinated swine, showing that the gum arabic is biocompatible in swine system testing. Additionally, when swine were slaughtered at market age (D152/173 FIG. 5) muscle and surrounding tissue was free of damage as determined by a veterinarian pathologist. The data provides the first evidence that gum arabic can be used as an emulsifier as a safe and efficacious adjuvant for killed viral and bacterial vaccines.

Application potentials include, but are not limited to, adjuvants that can be formulated with killed microbes and/or their subunit antigens in the forms of injections or microgel encapsulations, or other delivery approaches.

1. An oil-based adjuvant comprising a plant-derived surfactant, an aqueous component, and an oil.

2. The adjuvant of claim 1, wherein said plant-derived surfactant is nontoxic.

3. The adjuvant of claim 1, wherein said plant-derived surfactant is a water-soluble gum.

4. The adjuvant of claim 3, wherein said plant-derived surfactant is gum arabic.

5. The adjuvant of claim 1, wherein said adjuvant is in a form selected from the group consisting of water-in-oil, oil-in-water, and water-in-oil-in-water emulsions.

6. The adjuvant of claim 1, wherein said aqueous component is selected from the group consisting of water, buffered aqueous solutions, and mixtures thereof.

7. The adjuvant of claim 1, wherein said oil is derived from a plant or animal source.

8.-9. (canceled)

10. The adjuvant of claim 1, wherein said oil is a petroleum derivative.

11. The adjuvant of claim 1, wherein said oil comprises a mixture of different oils.

12. The adjuvant of claim 1, wherein said adjuvant is substantially free of saponin.

13. The adjuvant of claim 1, wherein said adjuvant is substantially free of stabilizers or emulsifiers other than said plant-derived surfactant.

14. (canceled)

15. The adjuvant of claim 1, said adjuvant consisting essentially of said plant-derived surfactant, aqueous component, and oil.

16. An immunogenic composition for eliciting an immunological response, said immunogenic composition comprising an adjuvant according to claim 1 and an active agent.

17. The immunogenic composition of claim 16, wherein said active agent is selected from the group consisting of killed virus, modified live virus, viral or bacterial proteins, viral or bacterial DNA, toxoids, protein subunits, tumor antigens, and combinations thereof.

18. The immunogenic composition of claim 16, wherein said active agent is an antigen derived from a pathogen and/or its cells, tissues, toxins, subunits, particles, nucleic acids, and/or surface proteins.

19. The immunogenic composition of claim 18, wherein said pathogen is:
(a) a virus selected from the group consisting of influenza, cholera, polio, hepatitis A, hepatitis B, HPV, rabies, canine distemper, measles, rubella, mumps, canine adenovirus Type 2, parainfluenza, coronavirus, and porcine respiratory and reproductive syndrome virus; or
(b) a bacteria selected from the group consisting of Mycoplasma hyopneumoniae, Bordetella bronchiseptica, Mycobacterium tuberculosis, Salmonella typhi, Staphylococcus aureus, and Streptococcus suis.

20-27. (canceled)

28. A vaccine system comprising:
an adjuvant according to claim 1; and
instructions for co-administering said adjuvant to a subject with an active agent.

29. (canceled)

30. The vaccine system of claim 28, further comprising an active agent, wherein said active agent and said adjuvant are mixed together in unit dosage form.

31. The system of claim 28, further comprising an active agent, wherein said active agent is in a first container and said adjuvant is in a second container separate from said active agent.

32. The system of claim 31, wherein said active agent is desiccated or freeze-dried.

33. (canceled)
